(19) United States
(12) Plant Patent Application Publication
(10) Pub. No.: US 2009/0193549 P1
(43) Pub. Date: Jul. 30, 2009

(54) CHESTNUT PLANT NAMED 'AU GOBBLER I'

(50) ;

(75) Inventors: W. Alfred Dozier, JR., Opelika, AL (US); J. D. Norton, Opelika, AL (US); Curtis J. Hansen, Opelika, AL (US)

Correspondence Address: HAVERSTOCK & OWENS LLP, 162 N WOLFE ROAD, SUNNYVALE, CA, 94086, US

(73) Assignee: Auburn University, an Alabama Corporation

(21) Appl. No.: 12/012,030

(22) Filed: Jan. 30, 2008

(51) Int. Cl. A01H 5/00

(52) U.S. Cl. PLT/152

(57) ABSTRACT

‘AU Gobbler I’ is a new and distinct Chinese chestnut cultivar that is precocious and produces a large crop annually of small nuts that mature early and start dropping about August 25. Nut drop continues for a 4 to 5 week period. The nuts mature and start dropping 3-4 weeks before most cultivars. The early nut drop and small nut size of ‘AU Gobbler I’ makes it ideal for use as a food source for turkey and other wildlife species. ‘AU Gobbler I’ is an excellent companion cultivar to ‘AU Gobbler II’ as nut drop begins about 10 days earlier than nut drop of ‘AU Gobbler II’ and extends the season of available food supply for turkey and other wildlife. The major nut drop period of ‘AU Gobbler I’ occurs before the major nut drop period of ‘AU' Gobbler II’.

### LATIN NAME OF THE GENUS AND SPECIES OF THE PLANT CLAIMED

Castanea P. Mill., Castanea mollissima Blume.

### VARIETY DENOMINATION

‘AU GOBBLER I’

### BACKGROUND OF THE INVENTION

A Chinese chestnut planting was established at Auburn University, Auburn, Ala., from nuts collected in China. The planting was established on the United States Department of Agriculture Horticulture Farm which in later years became the Mainstation Horticulture Farm. Precocious and prolific-bearing, blight resistant seedlings were selected for nut appearance, size and quality. Each generation of seedlings were the product of controlled mass pollination from the most promising seedlings selected from the previous generation. ‘AU Cropper’, ‘AU Leader’ and ‘AU Homestead’ were released from a second generation of approximately 2000 seedlings. A planting of third generation seedlings from controlled mass pollination of ‘AU Leader’, ‘AU Homestead’ and ‘AU Cropper’ was established at the Auburn University Piedmont Substation at Camp Hill, Ala.

### SUMMARY OF THE INVENTION

‘AU GOBBLER I’ is an open pollinated seedling of ‘AU Leader’.

The present invention relates to a new and distinct Chinese chestnut cultivar that is precocious, produces a heavy crop annually, average nut size is small (7.7 g) and begins nut drop about August 25 and continues for a 4 to 5 week period. The nuts mature and start dropping 3-4 weeks before most chestnut cultivars. The large crop of small nuts produced by ‘AU Gobbler I’ chestnut is an excellent food source for turkey and other wildlife. Its small nut size is ideal for consumption by turkey. The plant has not been affected by chestnut gall wasp or chestnut blight.

‘AU Gobbler I’ is an excellent companion cultivar to ‘AU Gobbler II’ as nut drop begins about 10 days earlier than nut drop of ‘AU Gobbler II’ and extends the season of available food supply for turkey and other wildlife. ‘AU Gobbler II’ is disclosed in U.S. patent application Ser. No. ______, filed on ______, and entitled “CHESTNUT PLANT NAMED ‘AU GOBBLER II’”, which is hereby incorporated by reference.

The new cultivar is able to be asexually reproduced by budding or grafting onto a seedling Chinese chestnut rootstock. The unique characteristics come true to form and are established and transmitted through asexual propagation.

### BRIEF DESCRIPTION OF THE DRAWINGS

FIG. 1 is a photograph of nuts of the ‘AU GOBBLER I’ cultivar.

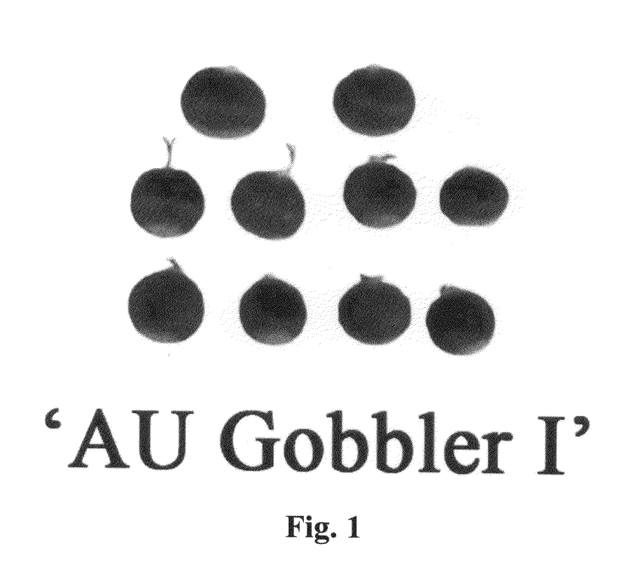

FIG. 2 is a photograph of nuts of the ‘AU GOBBLER I’ cultivar.

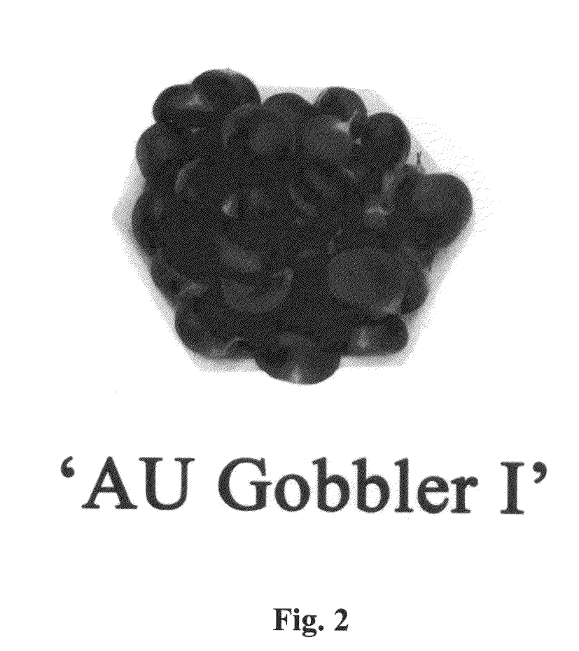

FIG. 3 is a photograph of a tree of the ‘AU GOBBLER I’ cultivar.

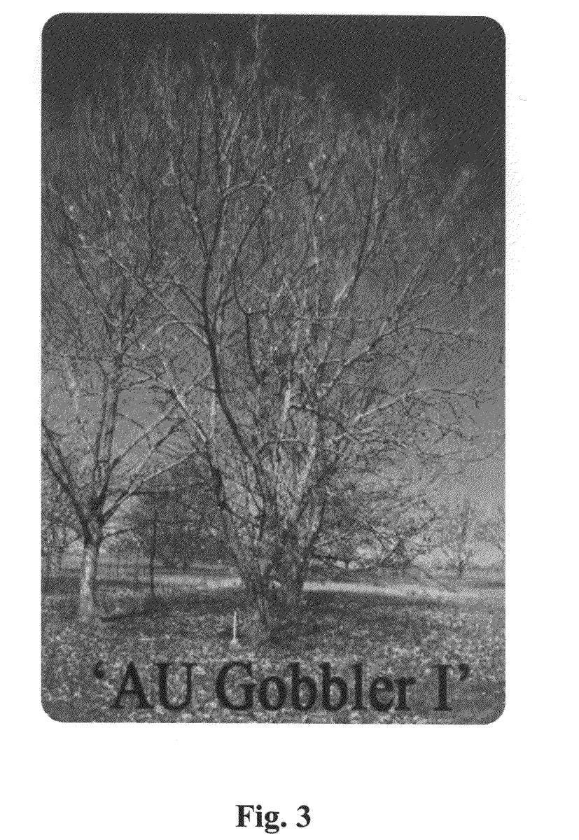

### DETAILED BOTANICAL DESCRIPTION

The Chinese chestnut, Castanea mollissima Blume, is a cold hardy temperate zone species native to China. It can be grown between 30° and 50° latitudes. The Chinese chestnut is resistant to chestnut blight fungus Cryphonectria parasitica. Generally, Chinese chestnuts are grown on a wide range of soils, but well-drained, deep and fertile soils are considered the best. Soils should be slightly acidic with pH 5.6-6.5. The name mollissima means soft hair and this species is recognized by dense hair on young leaves and downy yellow terminal parts of the shoots in winter. The leaf blade is thicker, and, in general, mature leaves are broader than those of other species. The nuts have a small scar or hilum. The pellicle of thin membranous skin on the nuts is thin and peels readily from the kernel. The trees are spreading type and long lived with a round top. The trees have bark with furrows and buds with 3-4 scales and leaves are 2 ranked, serrated with numerous parallel veins.

Chestnuts are monoecious and staminate flowers appear on erect cylindrical catkins with 10-20 stamens and 6-parted calyx. Pistillate flowers are borne on the lower part of the upper staminate catkins and rarely on separate catkins, usually 3 in a prickly symmetrical involucre with 7-9 styles and a 6-celled ovary. Nuts are small, brown with a pale scar at the base. Generally, 1-3 nuts per involucre or bur are present.

The ‘AU Gobbler I’ cultivar is a tall upright tree. The overall tree shape is obovate, with diffuse and upright branches. The original 15-year old ‘AU Gobbler I’ tree is 10.97 m tall, has a diameter at breast height of 35.9 cm, canopy width of 10.11 meters and covers an area of 80.24 square meters. The ‘AU Gobbler I’ produces a small nut that has a 5-year average weight of 7.7 grams. The average nut length is 1.04 inches (26.4 mm) and average nut width is 1.22 inches (31.0 mm). The nuts ripen early, start dropping by about August 25 and continue to drop over a 4 to 5 week period. The nuts mature and start dropping 3-4 weeks before most cultivars. The tree started producing nuts the third year after planting, is very prolific and produces annually.

The early nut drop and small nut size of ‘AU Gobbler I’ makes it ideal for use as a food source for turkey and other wildlife. The small nut is excellent for turkeys to use as a high energy food source at a time when food is scarce in native woodlands.

In the planting at the Pidemont Substation, Camp Hill, Ala., accurate yields could not be obtained due to extremely heavy wildlife (deer and turkey) feeding. Therefore, the trees were rated for crop load each year and ‘AU Gobbler I’ has been rated as annual bearer of large crops each season. In the fall of 2006, individual trees were caged with 6 foot (1.83 m) tall chicken wire prior to nut drop to exclude wildlife and nuts were picked up daily from the beginning through completion of nut drop. The original 15-year old ‘AU Gobbler I’ tree produced 127 pounds (57.73 kg) of nuts in 2006. This is a higher yield than that reported by similar aged trees.

The table below illustrates the specific differences between the ‘AU GOBBLER I’ cultivar and the ‘REVIVAL’ cultivar.

The botanical details of this new and distinctive variety of
chestnut tree - with color definitions (except those in
common color terms) referenced to Royal Horticultural
Society's Colour Chart (RHS) and color was also determined
using an electronic spectrophotometer to determine hue angle
and Chroma (spectrophotometer model CM-2002; Minolta Camera Co.,
Japan).
‘AU GOBBLER I’ CHESTNUT

Tree:
Size (at maturity) - large - fifteen year old original tree is 10.97 meters tall
with a canopy width of 10.11 meters and a canopy area of 80.24 sq.
meters
Tree shape is obovate.
Vigor - vexy vigorous
Trunk:
Form - upright, with branches high and diffuse
Texture - relatively smooth
Color of bark - greyed-green RHS 197A
Chroma C* 13.16, hue angle 77.24
Branches:
Form - upright
Texture relatively smooth
Lenticels - few, small
Branching habit - high and diffuse, spreading in upper regions of tree.
Color - new wood: brown, RHS N200A, Chroma C* 10.21,
hue angle 72.40; mature wood: greyed-green RHS 197A
Chroma C* 12.01, hue angle 88.37
Foliage:
Quantity - abundant
Density - dense
Leaves:
Size - large. Length (cm) 17.6 (14.2-24.3) [20]
width (cm) 6.5 (4.9-8.7) [20]
leaf ratio 2.8 (2.2-3.7) [20]
Shape - broadly elliptic to oblong
leaf tip - acute to occasionally acuminate
leaf base - rounded; oblique
Thickness- thick. Leaf venation 1° pinnate: 2° ± parallel,
prominent abaxially
Texture - moderately coriaceous (thick)
Margin - coarsely serrate; teeth spreading
Petiole - length 0.8cm (0.6-1.1) [20]
Petiole pubescence - glabrous to sparsely hairy
Color - adaxial surface, glabrous blade; glabrous veins, rarely with
sparse simple hairs, dark green; moderately shiny, RHS 147A,
Chroma C* 13.63, hue angle 122.39
abaxial surface, densely stellate hairs on blade; glabrous veins, light green
RHS 147B, Chroma C* 17.47, hue angle 112.24
Bloom:
Amount of bloom - heavy
Color - at anthesis, RHS 161D greyed-yellow group, RHS 157D
green-white group, RHS 155C white group
Blooming period - mid-May. After foliation in April.
Age at which tree starts flowering - early, 2-3 years after graft
replacement.
Male flower- Catkin length (cm) - 12.4 (9.4-15.4) [10]
Male flower - stamen number per catkin - 11.3 (10-13) [10]
Female flower - flower number per bur - 2.8
Female flower - style number per flower 8.4 (7-10) [11]
Crop:
Bearing - regular annual, bearer
Productivity - prolific
Ripening period - long, nuts begin dropping about August 25 and
continue for a 4-5 week period.
Distribution of nuts on tree - well distributed, fruits on terminals with
2-4 burs per terminal.
Tenacity - burs crack while on tree and nuts release and drop from bur.
Hull:
Description - spiny, round bur
Size - 1.8-2.3″ in diameter
Number of nuts - 2-3 per bur
Dehiscence - splits easily when still on tree
Color - yellow-green at dehiscence, RHS N144C
Nut:
Size - small; average size - 1.04″ × 1.22″ × 0.65″. Average
weight -7.7 grams - 59 (53-90) nuts per pound.
Form - flattened on 1 side, occ.2; mostly hemispheric on other side;
broadly rounded basally; rounded apically with a small tip.
Blossom end - small pointed tip
Basal end - flattened
Color - dark brown, lustrous; RHS 200B, Chroma C* 12.96, hue
angle 38.86
Pubescence - mixed long and short, fine white hairs densely covering
the distal ⅛ end of the nut, glabrous elsewhere
Shell - thin
Hardness of shell - relatively hard, yet not rigid
Texture of shell - smooth
Percentage of kernel to nut - very high-90-95%
Kernel:
Size - almost as large as nut size
Form - same as nut shape
Pellicle - thin
Flavor - excellent, very sweet
Color - light straw color, RHS 153D, Chroma C* 53.46, hue angle 82.55
Resistance to insects: no unusual susceptibilities noted
Resistance to disease: no susceptibilities to disease noted
The chestnut tree and its nuts herein described may
vary in slight detail due to climatic and soil conditions
under which the variety may be grown; the present
description being of the variety as grown in Camp Hill,
Ala.

The botanical details of this variety of
chestnut tree - with color definitions (except those in
common color terms) referenced to Maerz and Paul
Dictionary of Color - are as follows:
‘REVIVAL’

Tree:
Size (at maturity) - large
Vigor - very vigorous
Trunk:
Form - upright with branches spreading in upper
reaches of tree.
Texture - relatively smooth
Color of bark - Silvergray (13-A-1)
Branches:
Form - strong
Texture - relatively smooth
Lenticels - few, small
Branching habit - spreading in upper region of tree
Color - new wood: reddish brown and glossy, mature
wood: silver gray
Foliage:
Quantity - abundant
Density - dense
Leaves:
Size - large. Average length - 5-7″ (including petiole).
Average width - 24″
Shape - oblong with acute tip and rounded base
Thickness - thick
Texture - smooth
Margin - dentate
Petiole - length: medium. Thickness: medium.
Color - Top side - glossy dark green (22-L-12).
Under side - lighter green (21-D-7).
Bloom:
Amount of bloom - heavy
Color - cream white (17-B-1)
Blooming period - late. After leaf out in April
Age at which tree starts flowering - early; 2-3 years
years after graft replacement.
Crop:
Bearing - regular (yearly) bearer
Productivity - prolific
Ripening period - short. September 15-October 1.
Distribution of nuts on tree - well distributed
Tenacity - burrs crack while on tree and nuts easily
release, many falling by themselves
Hull:
Description - spiny, round burr
Size - 3-4″ in diameter
Number of nuts - 2-3 per burr
Dehiscence - splits easily when still on tree. Some
entire burrs split and fall to ground
Color - brown (15-A-8)
Nut:
Size - large. Average size - 1⅛″ × 1⅛″ × 1″ thick.
Average weight - 24-32 nuts per pound
Form - broad and ovoid on one side, flat on other side
Blossom end - pointed tip
Basal end - flattened
Color - India Red (7-L-6).
Shell - thin
Hardness of shell - relatively hard, yet not rigid
Texture of shell - smooth
Percentage of kernel to nut - very high (95%)
Kernel:
Size - almost as large as nut size
Form - same as nut shape
Pellicle - thin
Flavor - excellent. Very sweet.
Color - Oyster white (10-B-1)
Resistance to insects: no unusual susceptibilities noted
Resistance to disease: very high inherent resistance to
chestnut bark fungus (Endothia parastica), no
other susceptibilities to any other disease
The chestnut tree and its nuts herein described may
vary in slight detail due to climatic and soil conditions
under which the variety may be grown; the present
description being of the variety as grown in Alachua,
Fla.

What is claimed is:

1. A new and distinct cultivar of the species Castanea mollissima Blume named ‘AU GOBBLER I’ as described and illustrated herein.
